(19) United States
(12) Patent Application Publication
(10) Pub. No.: US 2026/0229362 A1
(43) Pub. Date: Aug. 6, 2026

(54) METHODS FOR AUTOMATED PROCESSING OF BIOMEDICAL DATA

(71) Applicant: INSTITUTE OF MEDICAL INFORMATION, CHINESE ACADEMY OF MEDICAL SCIENCES, Beijing (CN)

(72) Inventors: Sizhu WU, Beijing (CN); Zhengyong HU, Beijing (CN); Xiaolei XIU, Beijing (CN); Anran WANG, Beijing (CN)

(73) Assignee: INSTITUTE OF MEDICAL INFORMATION, CHINESE ACADEMY OF MEDICAL SCIENCES, Beijing (CN)

(21) Appl. No.: 19/632,441

(22) Filed: Mar. 30, 2026

Related U.S. Application Data
(63) Continuation-in-part of application No. 19/199,396, filed on May 6, 2025.

(30) Foreign Application Priority Data
May 14, 2024 (CN) 202410595015.0

(51) Int. Cl. G16H 50/20

(52) U.S. Cl. CPC G16H 50/20

(57) ABSTRACT

Provided is a method for automated processing of biomedical data, including: determining at least one candidate synonym pair from a biomedical dataset based on a knowledge graph; determining an original context of data elements in the candidate synonym pair; performing concurrent retrieval in a biomedical corpus based on the data elements to recall at least K groups of context fragments distributed across different application scenarios for each element; inputting the original context and context fragments into an LLM to obtain a semantic consistency score; determining a merged semantic group and generating a merged semantic label therefor; deploying the merged semantic label into a data reading plug-in; controlling the plug-in to perform a standardized payload conversion on a received heterogeneous service data stream: map heterogeneous character identifiers to a unified label appended to a data packet, and retain the heterogeneous character identifier as an original traceability field.

### CROSS-REFERENCE TO RELATED APPLICATIONS

This application is a Continuation-in-part of U.S. application Ser. No. 19/199,396, filed on May 6, 2025, which claims priority to Chinese Patent Application No. 202410595015.0, filed on May 14, 2024, the entire contents of each of which are hereby incorporated by reference.

### TECHNICAL FIELD

The present disclosure generally relates to the field of medical data processing technology, and in particular, to a method for automated processing of biomedical data.

### BACKGROUND

At present, biomedical data sharing can improve the efficiency of medical research and enhance the transparency of medical research. The academic field has also put forward mandatory requirements for research reproducibility and data disclosure, and an increasing number of medical researchers choose to disclose and even share original biomedical data. However, biomedical data have highly complex semantics, and are prone to problems such as synonymy and ambiguity. Meanwhile, shared biomedical data lack unified standards and specifications at the level of data fields or value domains, resulting in ambiguous data semantics and the inability to perform comparison and joint analysis between different datasets. For example, the English name of the field or variable “gender” in a dataset may be expressed as gender or sex; in terms of value domain, it may be directly expressed as “male” or “female” in text, or represented by numerical values 0 and 1, where 0 indicates male and 1 indicates female. Without unified data element names and value domain specifications, fields or variables with the same semantics in different datasets are unable to be integrated or jointly analyzed, and it is also difficult for researchers to understand data semantics and conduct analysis and utilization, which greatly hinders data sharing. Accordingly, metadata and data element standards for datasets are very important, which can standardize and unify data structures and semantic expressions.

However, most current data standards are published in unstructured forms such as PDF. Dataset standards in many clinical professional fields involve 200 to 300 data elements, and different data elements may define or use different value domains. At present, they may only support text search, reading, and understanding, making them difficult to effectively utilize during metadata creation, with poor machine readability and processability, which is also why the standards are difficult to apply and implement.

Therefore, how to improve machine readability and semantic interoperability on the basis of enhancing the availability and utilization rate of metadata, data element, classification and value domain standards for domain datasets is an urgent problem to be solved by those skilled in the art.

### SUMMARY

In view of this, the present disclosure provides a method for automated processing of biomedical data, performed by a computing system including a processor and a memory, including: determining, based on a knowledge graph, at least one candidate synonym pair from a biomedical dataset to be analyzed, wherein a topological distance between any two data elements of the at least one candidate synonym pair in the knowledge graph is less than or equal to a topological distance threshold; determining an original context of data elements in the at least one candidate synonym pair based on the biomedical dataset to be analyzed; performing a concurrent retrieval in a biomedical corpus based on the data elements, to recall at least K groups of context fragments distributed across different application scenarios for each of the data elements; inputting the original context and the at least K groups of context fragments of the data elements included in the at least one candidate synonym pair into a large language model (LLM) to obtain a semantic consistency score that measures a semantic similarity degree of the at least one candidate synonym pair, wherein the LLM is a machine learning model; determining a merged semantic group that is semantically equivalent from the at least one candidate synonym pair based on the semantic consistency score, and generating a merged semantic label for the merged semantic group; dynamically deploying the merged semantic label into a data reading plug-in located between different biomedical systems; and controlling the data reading plug-in to perform a process including: in response to receiving a heterogeneous service data stream containing a heterogeneous character identifier, performing a standardized payload conversion on the heterogeneous service data stream based on the merged semantic label: mapping the heterogeneous character identifier to a unified merged semantic label and appending the unified merged semantic label to a data packet; and retaining the heterogeneous character identifier as an original traceability field.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a flowchart illustrating an exemplary method for automated processing of biomedical data according to some embodiments of the present disclosure;

FIG. 2 is a flowchart illustrating an exemplary process for constructing a knowledge graph according to some embodiments of the present disclosure;

FIG. 3 is a schematic diagram illustrating a knowledge model according to some embodiments of the present disclosure;

FIG. 4 is a schematic diagram illustrating a data instance of a portion of a knowledge graph according to some embodiments of the present disclosure; and

FIG. 5 is a schematic diagram illustrating relationships among a dataset standard, a data element set, a data element, and a value domain code according to some embodiments of the present disclosure.

### DETAILED DESCRIPTION

The technical solutions in the embodiments of the present disclosure will be clearly and completely described below in conjunction with the accompanying drawings in the embodiments of the present disclosure, and it is clear that the embodiments described are only a portion of the embodiments of the present disclosure, and not all of the embodiments. Based on the embodiments in the present disclosure, all other embodiments obtained by a person of ordinary skill in the art without making creative labor fall within the scope of protection of the present disclosure.

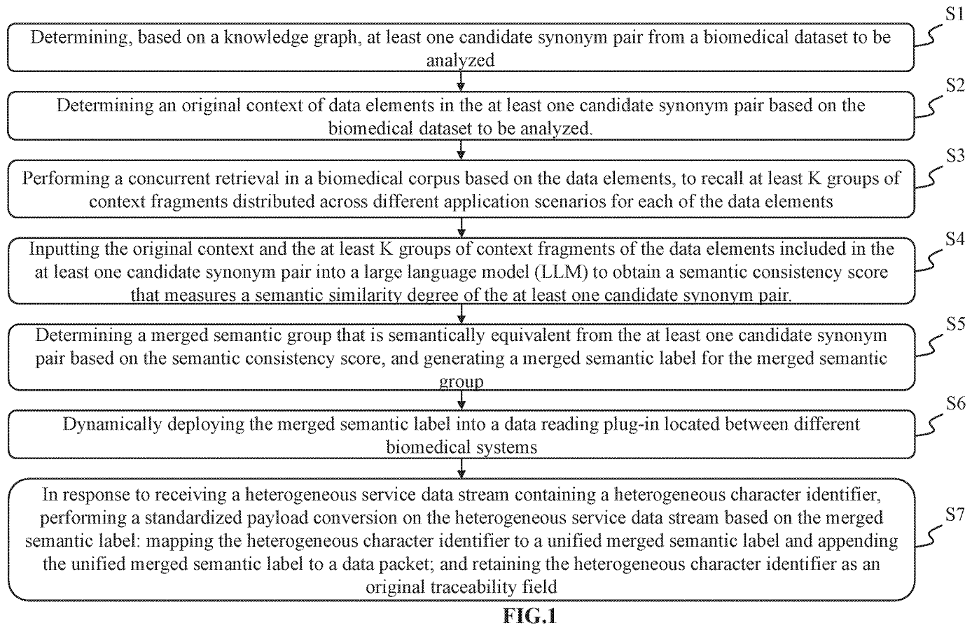

FIG. 1 is a flowchart illustrating an exemplary method for automated processing of biomedical data according to some embodiments of the present disclosure. The method for automated processing of biomedical data may be performed by a computing system including a processor and a memory. As shown in FIG. 1, the method for automated processing of biomedical data includes S1-S7.

S1, determining, based on a knowledge graph, at least one candidate synonym pair from a biomedical dataset to be analyzed.

The biomedical dataset to be analyzed refers to a collection composed of various kinds of data in a biomedical field. For example, the biomedical dataset may include various forms of data collected in processes such as biomedical research, clinical practice, and health monitoring.

In some embodiments, for biomedical systems deployed in different application scenarios (such as hospital HIS systems and laboratory LIS systems), the biomedical datasets used thereby usually have different data sources, and there are heterogeneous differences in data encoding and format rules. Therefore, unification needs to be performed through the method for automated processing of biomedical data of the present disclosure.

In some embodiments, the biomedical dataset may include a plurality of data elements.

The data element is the smallest data unit having an independent meaning in a specific business context, and is a core basic carrier for developing data standardization and achieving cross-system data interoperability in a biomedical field.

In some embodiments, for biomedical datasets from different data sources, data elements may correspond to different semantic forms. For example, in a disease diagnosis and treatment scenario, a data element may serve as a standardized storage identifier and configured to establish mapping associations with medical scales and diagnostic terms, support the standardized circulation of diagnosis and treatment evaluation results, and assist in the assessment of patient disease severity. As another example, in a laboratory testing scenario, a data element may correspond to a testing item name, an indicator code, an evaluation result, or the like.

The knowledge graph refers to a data structure that organizes data elements in the form of nodes and relationships. The knowledge graph can help a user better understand the semantic associations, hierarchical affiliations, and mapping relationships between data elements. The knowledge graph may also be referred to hereinafter as the biomedical science dataset standard data element knowledge graph.

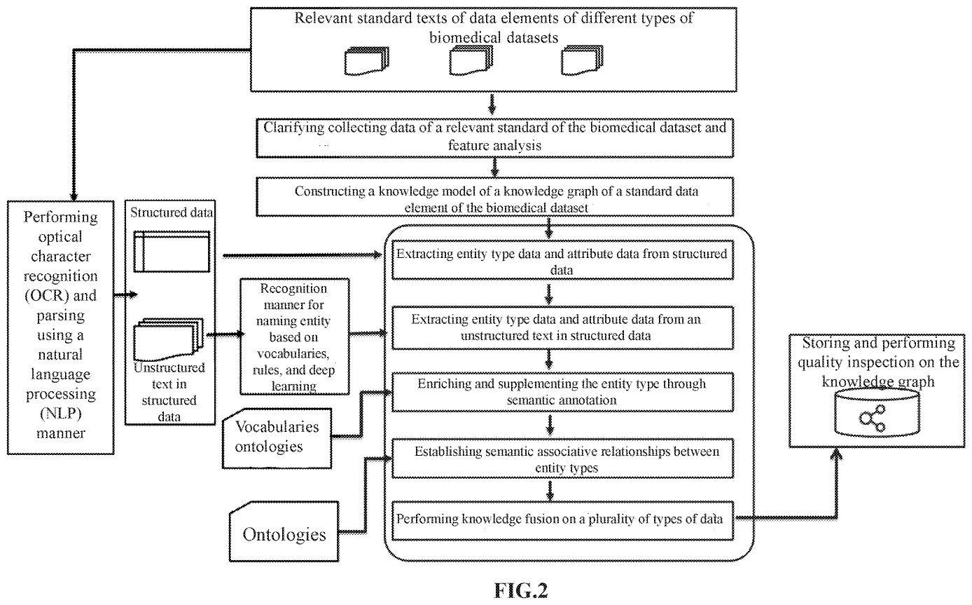

In some embodiments, the knowledge graph is composed of at least one node and at least one edge. The nodes correspond to data elements, and a node attribute may include information such as a biomedical system to which the data element belongs, an internal identifier, a data element name, and an ontology category. The edges correspond to semantic associative relationships between the nodes, and an edge attribute may include information such as relationship attributes. More descriptions of the ontology category and internal identifier in the node attributes, may be found in FIG. 2 and related descriptions thereof.

In some embodiments, in the knowledge graph, the closer two nodes are, the higher the semantic similarity degree of the corresponding two data elements. When there are relationships such as synonymy, a hierarchical relationship, or an associative reference between the two data elements, the two corresponding nodes are connected by an edge.

For example, the knowledge graph may include nodes such as hypertension, nifedipine, and cardiovascular medicine. The hypertension node is connected to the nifedipine node, and the edge attribute is treatment; the hypertension node is connected to the cardiovascular medicine node, and the edge attribute is visit department.

In some embodiments, the processor may construct the knowledge graph in a plurality of manners. For example, the processor may import the biomedical dataset to be analyzed into graph storage engines such as a JanusGraph distributed graph database and a Virtuoso RDF triple store. The graph storage engines automatically complete the mapping construction of entity nodes and associative relationships.

In some embodiments, the processor may further obtain structured data and an unstructured text of each multivariate standard text in a multi-source standard text set; extract an entity type and attribute data from the structured data and the unstructured text; and perform knowledge fusion on the entity type and the attribute data based on a semantic associative relationship to generate the knowledge graph. More descriptions of the knowledge graph construction may be found in FIG. 2 and related descriptions thereof.

The candidate synonym pair is a group of data elements that are semantically potentially equivalent. For example, gender and sex chromosome in the biomedical dataset may both reflect the gender of a patient; therefore, the data element “male” and the data element “XY” are a candidate synonym pair.

In some embodiments, a candidate synonym pair needs to have the same ontology category. The ontology category reflects the type to which a data element belongs. For example, the ontology categories include but are not limited to testing items, drug terms, or disease diagnoses.

In some embodiments, for a candidate synonym pair, a topological distance between any two data elements of the candidate synonym pair in the knowledge graph is less than or equal to a topological distance threshold.

The topological distance is the minimum count of edges traversed from one node to another node. The topological distance between two directly adjacent nodes (i.e., directly connected by an edge) is 1.

In some embodiments, the processor may randomly select a node A from the knowledge graph that has not been determined to belong to a candidate synonym pair. All nodes that belong to the same ontology category as the node A and whose topological distance is less than or equal to the topological distance threshold are collectively taken as a candidate synonym pair with the node A. The processor repeats selecting the remaining nodes that have not been determined to belong to a candidate synonym pair and performs the above operations, until all data elements in the knowledge graph have completed their candidate synonym pair division, or until there are no matching nodes to form a valid candidate synonym pair. The topological distance threshold may be preset based on domain prior experience.

In some embodiments, the processor may further determine the node density and the connection complexity of the ontology category corresponding to each of the data elements based on the knowledge graph; and determine the topological distance threshold corresponding to the data element based on the node density and the connection complexity. Data elements of different ontology categories have different topological distance thresholds.

The node density is used to reflect a distribution density of the nodes of the same ontology category in the knowledge graph. The higher the node density, the higher the semantic similarity degree between corresponding nodes.

In some embodiments, the node density is positively correlated with a total count of nodes of the same ontology category, and is negatively correlated with a distribution area of the nodes of the same ontology category in the knowledge graph.

In some embodiments, the processor divides the knowledge graph into a plurality of local subgraphs by using graph cutting algorithms such as METIS. For a local subgraph i, the processor counts a sub-area Sᵢ of the local subgraph i and a count of nodes (Γₐ, Γ_{b}, Γ_{c}) of different ontology categories (a, b, c), and further determines the node densities

$$( \frac{{Γ}_{a}}{{S}_{i}} , \frac{{Γ}_{b}}{{S}_{i}} , \frac{{Γ}_{c}}{{S}_{i}} )$$

of the nodes of different ontology categories in the local subgraph i. The processor determines an average of the node densities of nodes of a certain ontology category in all local subgraphs, as the node density of the ontology category.

For example, the processor may determine the node density ηₐ of the ontology category a by using formula as follows:

$${η}_{a} = \frac{1}{n} \times {\sum }_{i = 1}^{n} \frac{{Γ}_{a}}{{S}_{i}}$$

where ηₐ represents the node density of the ontology category a, n represents the total count of local subgraphs i, Γₐ represents the count of nodes of the ontology category a within the local subgraph i, and Sᵢ represents the sub-area Sᵢ of the local subgraph i.

The connection complexity refers to a complexity of edges connecting nodes of the same ontology category in the knowledge graph. The greater the connection complexity, the more disordered the relationships among the nodes of the ontology category.

In some embodiments, for all nodes of an ontology category, the processor determines a count of edge types extending outwards from the nodes and a total count of edges. For example, if a hypertension node is connected to a nifedipine node, with an edge attribute being “treatment”; and the hypertension node is connected to a cardiovascular medicine node, with an edge attribute being “department of visit”, then both the count of edge types extending outwards from the hypertension node and the total count of edges are 2.

In some embodiments, the connection complexity is positively correlated with the count of edge types and the total count of edges connected to the nodes of the ontology category.

In some embodiments, based on the node density and the connection complexity, a topological distance threshold corresponding to a data element is determined.

The topological distance threshold refers to a determining condition for whether two data elements are a candidate synonym pair.

In some embodiments, the processor determines the topological distance threshold based on a pre-constructed first vector database.

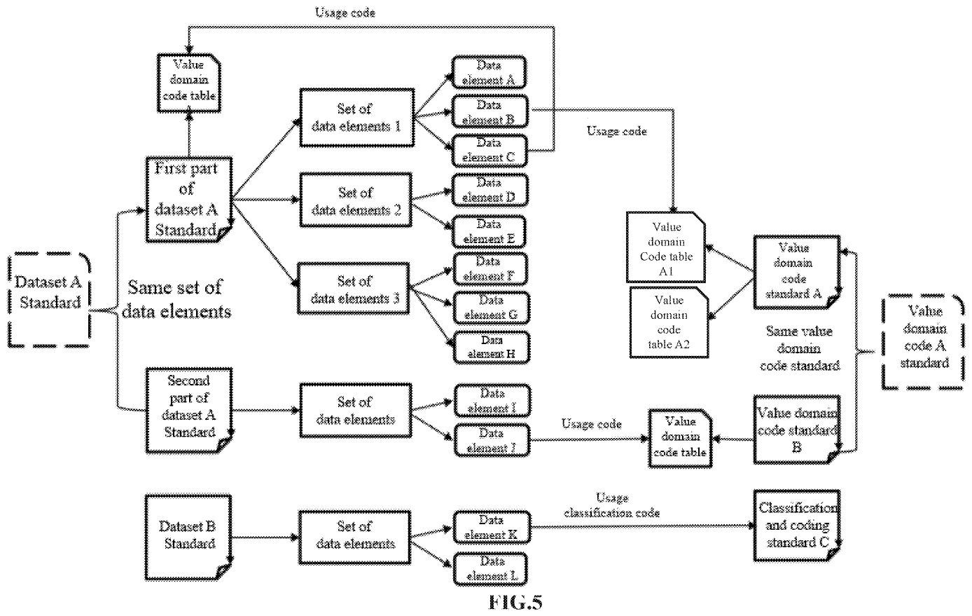

In some embodiments, for nodes of an ontology category, the processor may construct a target vector based on the node density and the connection complexity of the nodes of the ontology category; perform vector matching in the first vector database based on the target vector to determine a historical feature vector with the highest similarity; and determine a label corresponding to the historical feature vector with the highest similarity as the topological distance threshold corresponding to a data element under a current ontology category. The first vector database may include a plurality of historical feature vectors with labels. The processor constructs the historical feature vectors based on historical node densities and historical connection complexities corresponding to nodes of different ontology categories in historical data. In a plurality of merges corresponding to the historical feature vectors, a plurality of historical topological distance thresholds were used to determine the merged semantic groups. The processor selects the historical topological distance threshold that may obtain the largest count of merged semantic groups, as the label of the historical feature vector. More descriptions of the merged semantic group may be found in FIG. 5 and related descriptions thereof.

In some embodiments, based on the node density and the connection complexity, a topological distance threshold corresponding to a data element is determined, and the topological distance threshold is dynamically and adaptively adjusted through local structural features of the knowledge graph, which effectively reduces the risk of missing recall or false recall of the candidate synonym pairs caused by different degrees of subdivision of specific medical concepts.

S2, determining an original context of data elements in the at least one candidate synonym pair based on the biomedical dataset to be analyzed.

The original context refers to original information of the data element in the biomedical dataset.

In some embodiments, if a data element originates from the structured data, the original context includes a table name, a column name, and content of adjacent fields of a database where the data element is located; if a data element originates from the unstructured text, the original context includes a sentence or a paragraph where the data element is located in the unstructured text.

In some embodiments, for a data element originating from structured data, the processor reads metadata information of the database to which the data element belongs, extracts a table name where the data element is located and the column name of a column to which the data element belongs, and obtains content of a preset count of adjacent fields before and after the column to combine and form the original context of the data element. For a data element originating from unstructured text, the processor first locates a distribution location of the data element in the unstructured text, and intercepts a complete sentence or a complete paragraph containing the data element, as the original context of the data element.

S3, performing a concurrent retrieval in a biomedical corpus based on the data elements, to recall at least K groups of context fragments distributed across different application scenarios for each of the data elements.

The biomedical corpus refers to a database containing a large amount of textual data in the biomedical field. For example, the biomedical corpus includes, but is not limited to, medical literature, clinical diagnosis and treatment guidelines, electronic medical record samples, or the like.

The application scenarios refer to specific business environments to which data elements are applicable. The application scenarios include but are not limited to a medical record scenario, a laboratory report scenario, a medical research scenario, an academic scenario, or the like. Since semantic expressions and associative pointing of the same biomedical data element may differ in different application scenarios, recalling context fragments in different application scenarios can further improve the accuracy of subsequent synonym determination.

The concurrent retrieval refers to a retrieval manner capable of performing a plurality of information retrievals concurrently. The processor may, based on high parallel computing features of a graphics processing unit (GPU), decompose a retrieval request of each data element in different application scenarios into mutually independent retrieval sub-tasks, and map the retrieval sub-tasks to a plurality of parallel computing cores of the GPU for synchronous execution, so as to achieve the concurrent retrieval.

In some embodiments, the processor may perform the concurrent retrieval in a plurality of manners. For example, the processor may, through a full-text search engine such as ElasticSearch, concurrently initiate a plurality of query requests for literal texts of the data element in the biomedical corpus, to quickly locate all text locations containing the data element. By taking a matching location as a center, the processor may intercept textual content of a preset length before and after the text location to obtain candidate fragments of the data element.

In some embodiments, since the biomedical corpus is large in volume, the same data element may be matched to thousands of the candidate fragments, a large amount of which only incidentally mention the data element and have extremely low actual semantic relevance to the data element; if directly included in subsequent analysis, a large amount of invalid interference information would be introduced. The processor may count an occurrence frequency of the data element in each candidate fragment, sort candidate fragments in descending order based on the occurrence frequency, select top K groups of candidate fragments according to the sorting result, take the top K groups of candidate fragments as the recalled context fragments, so as to greatly reduce the interference of invalid information on subsequent synonym determination, improve the determination accuracy. The value of K may be determined based on prior experience.

In some embodiments, the concurrent retrieval includes a sparse retrieval and a dense retrieval. In some embodiments, performing the concurrent retrieval in the biomedical corpus based on the data elements includes: performing the sparse retrieval on the biomedical corpus to obtain a sparse fragment set; constructing a data element vector and a corpus vector based on the data elements and a corpus text; performing the dense retrieval based on the data element vector and the corpus vector to obtain a dense fragment set; and performing a fusion ranking based on the sparse fragment set and the dense fragment set to recall the at least K groups of context fragments for each of the data elements.

The sparse retrieval refers to a retrieval technology for exact matching. The processor may perform the sparse retrieval by adopting an algorithm such as Best Matching 25 (BM25) and term frequency-inverse document frequency (TF-IDF). The processor takes the data elements to be retrieved and the biomedical corpus as inputs of the above-mentioned sparse retrieval algorithm, performs the sparse retrieval, and designate a set of text fragments finally output as the sparse fragment set.

The dense retrieval refers to a retrieval technology for fuzzy matching. The dense retrieval converts the data elements to be retrieved and the corpus text into high-dimensional vectors, and achieves semantic matching by determining similarity between the vectors to finally obtain the dense fragment set.

The difference between the dense retrieval and the sparse retrieval lies in that the sparse retrieval has a fast retrieval speed, but may only recall text fragments explicitly containing the data elements to be retrieved; while the dense retrieval completes recall through similarity matching of semantic vectors, is not limited by literal expressions, and may cover text fragments that do not literally contain the data elements to be retrieved, but are semantically highly relevant.

The corpus text refers to textual content included in the biomedical corpus.

The data element vector refers to a data element that has been vectorized. The corpus vector refers to the corpus text that has been vectorized. Since data elements and the corpus text are discrete text fragments and are unable to be directly used for the dense retrieval, vectorization processing is required to map the discrete text fragments to a high-dimensional mathematical space, to obtain continuous vectors, thereby facilitating the dense retrieval.

In some embodiments, before performing vectorization processing, the data elements and the corpus text need to undergo pre-processing. For the data element, the processor extracts information such as a biomedical system to which the data element belongs, an internal identifier, a data element name, and the ontology category, and concatenates all the above extracted information into a coherent short text, which serves as an input text for vectorization. The processor performs segmentation processing on the corpus text, and divides the corpus text into a plurality of corpus text fragments according to a preset word number window or natural paragraph boundaries.

After completing the above-mentioned pre-processing operations, by invoking a feature extraction model such as Sentence-BERT (SBERT) and minimal language model (MiniLM), the processor uses the coherent short texts corresponding to the data elements as inputs of the feature extraction model, and designates the high-dimensional vectors finally output are the data element vectors. The processor also uses the plurality of segmented corpus text fragments as inputs of the feature extraction model, and designates the high-dimensional vectors finally output are the corpus vectors.

The dense fragment set refers to a set of text fragments obtained by the dense retrieval.

In some embodiments, for any data element vector, the processor iterates to determine a cosine distance between the data element vector and each corpus vector, and determines a cosine similarity based on the cosine distance. The cosine similarity is negatively correlated with the cosine distance.

The processor forms a dense fragment set corresponding to the data element from text fragments corresponding to corpus vectors whose cosine similarity is greater than a preset similarity threshold.

The fusion ranking refers to a processing operation that merges and organizes the sparse fragment set and the dense fragment set to obtain at least K groups of context fragments.

In some embodiments, the processor may perform the fusion ranking in a plurality of manners. For example, if a total count of fragments included in the sparse fragment set is greater than K, and standard deviations of the cosine similarities between all fragments in the sparse fragment set and the data element is less than or equal to a preset standard deviation threshold, which indicates that the fragments recalled by the sparse retrieval exhibit sufficient literal matching and stable quality, the processor directly selects the top K fragments with the highest cosine similarity from the sparse fragment set to obtain the at least K groups of context fragments. By referring to the operations of the dense retrieval described above, the processor may respectively convert all fragments within the sparse fragment set into sparse fragment vectors through the vectorization processing and the feature extraction model. Then, the processor iterates and determines the cosine distance between the data element vector and each sparse fragment vector, and subsequently determines the cosine similarities of all fragments within the sparse fragment set.

If the total count of fragments included in the sparse fragment set is less than or equal to K, or if the standard deviation of the cosine similarities between all fragments in the sparse fragment set and the data element is greater than the preset standard deviation threshold, which indicates that the fragments recalled by the sparse retrieval are insufficient in number or have large fluctuations in matching quality, it is necessary to further supplement the context fragments by combining with the dense fragment set. The processor merges the top K/2 fragments with the highest cosine similarity from the sparse fragment set and the top K/2 fragments and the highest cosine similarity from the dense fragment set to obtain the at least K groups of context fragments.

In some embodiments of the present disclosure, by combining the sparse retrieval and the dense retrieval, the sparse retrieval helps ensure that fragments containing keywords are preferentially recalled, while the dense retrieval can supplement important semantically related contexts with different wordings. This hybrid retrieval manner can significantly improve the accuracy and the breadth of semantic coverage of the recalled context fragments.

In some embodiments, for each candidate synonym pair, the processor may determine a topological distance set of the data elements in the candidate synonym pair in the knowledge graph based on the knowledge graph; and determine a number K of groups of context fragments required to be recalled for each of the data elements in the candidate synonym pair based on the topological distance set.

The topological distance set refers to a set of topological distances between any two data elements in the candidate synonym pair. More descriptions of the topological distance may be found in above descriptions.

In some embodiments, based on the topological distance set, the processor determines a statistical value (for example, a mean, a median, etc.) of the topological distances of the data elements in the candidate synonym pair; and obtains the number K of groups of context fragments required to be recalled for each of the data elements in the candidate synonym pair by consulting the pre-constructed first preset table based on the statistical value of the topological distances.

The first preset table includes a correspondence relationship between a statistical value of historical topological distances and a historical number K of groups. The processor collects a large amount of historical data to construct the first preset table. Each piece of historical data includes the statistical value of historical topological distances and the historical number K of groups adopted during historical concurrent retrieval. The correspondence relationship between the statistical value of historical topological distances and the historical number K of groups is as follows: the statistical value of historical topological distances is positively correlated with the historical number K of groups.

When querying the first preset table, the processor uses a current statistical value of the topological distances as a matching basis, retrieves a statistical value of historical topological distances with the highest similarity from the first preset table, and determines a historical number K of groups corresponding to the statistical value of historical topological distances as a current number K of groups. The processor may determine the similarity through a cosine similarity approach or other algorithms.

In some embodiments, by dynamically adjusting the number K of groups according to the topological distances of the data elements in the knowledge graph, for candidate synonym pairs with close semantic association, using a smaller number K of groups helps save computing resources; for candidate synonym pairs with distant semantic relationship, using a larger number K of groups helps recall more sufficient context information for judgment.

In some embodiments, the fusion ranking further includes: clustering the sparse fragment set and the dense fragment set based on the different application scenarios to obtain at least one scenario cluster; for each scenario cluster of the at least one scenario cluster, sorting fragments within the scenario cluster according to a similarity between each of the fragments and a corresponding data element; determining fragments to be merged from a sorting result of each scenario cluster based on a preset selection ratio; and combining the fragments to be merged from all scenario clusters to obtain the context fragments.

The scenario cluster refers to a set formed by clustering context fragments having similar background features based on the application scenario. The fragments within the same scenario cluster have a high semantic similarity. More descriptions of the application scenario, may be found in the above operation S3.

In some embodiments, the processor may perform clustering based on density-based spatial clustering of applications with noise (DBSCAN), K-Means, or other clustering algorithms. The processor uses all fragments included in the sparse fragment set and the dense fragment set as an input for the clustering algorithm and uses the application scenario of the data element as a clustering constraint condition to obtain at least one scenario cluster output by the clustering algorithm.

In some embodiments, for each scenario cluster, the processor determines the corpus vectors corresponding to the fragments within the scenario cluster and the data element vector corresponding to the data element. The processor determines the cosine similarity between each of the corpus vectors and the data element vector based on the corpus vectors and the data element vector. The fragments are sorted in descending order according to the cosine similarity. More descriptions of the corpus vector, the data element vector, and the cosine similarity may be found in above descriptions.

The preset selection ratio refers to a proportion of fragments to be merged filtered and obtained from the respective scenario clusters. The fragments to be merged refer to fragments filtered and obtained from the respective scenario clusters. After the fragments to be merged are combined, the context fragments may then be obtained. The higher the preset selection ratio, the greater the count of fragments selected as fragments to be merged selected in a single scenario cluster, and the greater the count of obtained context fragments. The lower the preset selection ratio, the fewer the count of fragments selected as fragments to be merged selected in a single scenario cluster, and the lower the overall redundancy of the finally obtained context fragments.

In some embodiments, the processor may flexibly adopt preset selection ratios of different scales based on actual scenario needs. For example, for data collation, data mining, and other tasks having higher accuracy requirements but tolerating higher latency, the processor may adopt a higher preset selection ratio to obtain larger scale context fragments, to cover a plurality of application scenarios as much as possible. For emergency data processing, clinical inquiry, and other low-latency tasks, the processor may adopt a lower selection ratio to quickly obtain smaller scale context fragments, to improve the processing speed of the computing system.

In some embodiments, when P scenario clusters are obtained, and it is necessary to recall at least K groups of context fragments distributed across different application scenarios, the processor, based on a preset selection ratio M %, respectively selects the top M % ranked fragments from the sorting result of each scenario cluster as the fragments to be merged, and combines the fragments to be merged selected from all scenario clusters to form the final at least K groups of context fragments.

In some embodiments, to ensure that the count of the context fragments is greater than or equal to K, the preset selection ratio M % satisfies:

$$M \% \geq \frac{K}{( {N}_{1} + {N}_{2} + … + {N}_{P} )} \times 100 \% ,$$

where N_{P} denotes a count of fragments included in a P-th scenario cluster.

In some embodiments, introducing scenario clustering, which helps ensure the diversity and balance of the finally recalled context fragments among different application scenarios, can avoid the problem that recall results are dominated by a single scenario due to excessive corpus for a specific scenario.

S4, inputting the original context and the at least K groups of context fragments of the data elements included in the at least one candidate synonym pair into a large language model (LLM) to obtain a semantic consistency score that measures a semantic similarity degree of the at least one candidate synonym pair.

The LLM refers to a machine learning model trained on large-scale text data and capable of generating or understanding the meaning of language text. The LLM includes but is not limited to any one or a combination of publicly available models such as BERT, LLAMA, GPT, or customized model structures.

In some embodiments, an input for the LLM is original context of all data elements in a candidate synonym pair and at least K groups of context fragments, and an output of the LLM is a semantic consistency score among the data elements.

The semantic consistency score refers to an indicator that quantitatively evaluates the degree of equivalence of all data elements in a candidate synonym pair in terms of medical semantic connotation and referential entities. For example, the semantic consistency score is within a range of [0, 1], and the greater the semantic consistency score, the more semantically equivalent the data elements in the candidate synonym pair.

In some embodiments, to obtain the semantic consistency score, the original context of all data elements and the at least K groups of context fragments in the candidate synonym pair may be input to the LLM, and a determination instruction may be provided to the LLM in natural language. The processor determines a numerical value output by the LLM to be the semantic consistency score.

The determination instruction instructs the LLM to evaluate a degree of equivalence of the data elements of the candidate synonym pair at the biomedical semantic level based on the input, and finally output a corresponding quantitative value as the semantic consistency score. For example, the determination instruction may be: “Based on the input original context and context fragments of a plurality of data elements, determine a degree of approximation of the plurality of data elements in the biomedical field and output a corresponding numerical value, wherein the numerical value is within the range of [0, 1], the higher the similarity, the closer the numerical value is to 1, and the numerical value is 1 if completely identical.”

S5, determining a merged semantic group that is semantically equivalent from the at least one candidate synonym pair based on the semantic consistency score, and generating a merged semantic label for the merged semantic group.

The merged semantic group refers to a candidate synonym pair that refers to the same medical concept. For example, for the candidate synonym pair [“patient gender”, “patient_sex”, “Gender_Code”], all refer to gender. Therefore, the candidate synonym pair may be determined as the merged semantic group.

In some embodiments, since the data elements included in the candidate synonym pair only represent a relatively high association probability, they do not necessarily refer to the same medical concept. For example, the topological distance between “systolic pressure” and “blood pressure value” is relatively small, so these two data elements may be classified into the same candidate synonym pair, but the two are not equivalent concepts. Therefore, the processor further needs to perform a secondary screening on the candidate synonym pair based on the semantic consistency score. Only when all data elements in a candidate synonym pair refer to the same medical concept, the candidate synonym pair can be determined as the merged semantic group.

In some embodiments, the processor may determine the merged semantic group in a plurality of manners. For example, when the semantic consistency score of a certain candidate synonym pair is greater than or equal to a consistency threshold, the candidate synonym pair is determined as the merged semantic group. The consistency threshold may be preset based on prior experience.

In some embodiments, in response to the semantic consistency score of any candidate synonym pair being within a fuzzy interval, the processor obtains an associated feature of the data elements corresponding to the candidate synonym pair based on the knowledge graph, and determines whether the candidate synonym pair is the merged semantic group based on the associated feature.

The fuzzy interval refers to a preset numerical range of the semantic consistency score. When the semantic consistency score of a certain candidate synonym pair falls into the fuzzy interval, it indicates that the processor is unable to accurately determine whether the data elements in the candidate synonym pair refer to the same medical concept solely by the semantic consistency score output by the LLM, and further needs to make a determination in combination with other features.

The associated feature refers to structural relationships between a plurality of nodes in the knowledge graph. In some embodiments, the associated feature includes a count of associated nodes between any two nodes and an associated relationship between any two nodes.

The count of associated nodes refers to a total count of common parent nodes, child nodes, and sibling nodes shared by two nodes in the knowledge graph. The greater the count of associated nodes, it indicates that the higher the overlap of application scenarios of the data elements represented by the two nodes, and the greater the probability of semantic equivalence.

The associated relationship refers to whether the two nodes belong to strong association or weak association in the knowledge graph. The processor determines the associated relationship between the two nodes based on the edge attribute of the shortest path between the two nodes.

For example, when the edge attribute is “containment/belonging”, “synonymous/reuse”, “substitution”, or “self-enumerated reference type/enumerated definition type/enumerated reference type”, the edge is a strong association edge; and when the edge attribute is “reference/citation” or “involvement/relevance/association”, the edge is a weak association edge. If the shortest path between the two nodes consists entirely of strong association edges, then the two nodes belong to the strong association. If the shortest path between the two nodes includes a weak association edge, then the two nodes belong to the weak association. More descriptions of the edge attribute may be found in the description of the knowledge graph in operation S1.

In some embodiments, in the knowledge graph, if the count of associated nodes between the two nodes is greater than or equal to a preset count threshold, and the associated relationship belongs to the strong association, then the two data elements corresponding to the two nodes have a highly semantic consistency score, and the two data elements may be regarded as a merged semantic pair. Otherwise, the two data elements are regarded as non-merged semantic pairs.

In some embodiments, the processor determines a count of merged semantic pairs and a count of non-merged semantic pairs in a candidate synonym pair. If the count of merged semantic pairs is greater than the count of non-merged semantic pairs, then the candidate synonym pair is determined as the merged semantic group; if the count of merged semantic pairs is less than or equal to the count of non-merged semantic pairs, then the candidate synonym pair does not belong to the merged semantic group.

In some embodiments, by introducing the associated features of the knowledge graph for secondary determination when the semantic consistency score of the candidate synonym pair is within the fuzzy interval, the accuracy and reliability of merged semantic group identification in scenarios of fuzzy determination can be significantly improved.

The merged semantic label refers to a unique digital label assigned to the merged semantic group.

In some embodiments, the merged semantic label includes codes from authoritative medical terminology systems, e.g., the standardized codes from the systematized nomenclature of medicine-clinical terms (SNOMED CT) and the unified medical language system (UMLS). The merged semantic label may also include a custom-generated universally unique identifier (UUID).

In some embodiments, the processor may generate the merged semantic label for the merged semantic group in a plurality of manners. For example, if the data elements included in the merged semantic group have matching authoritative standard concepts, the processor may retrieve a standard term corresponding to the merged semantic group from terminology sets such as SNOMED CT and UMLS, and use the official unique encoding of the standard term as the merged semantic label for the merged semantic group. If the merged semantic group has no matching standard term, the processor may generate a unique UUID by itself as the merged semantic label for the merged semantic group.

S6, dynamically deploying the merged semantic label into a data reading plug-in located between different biomedical systems.

The data reading plug-in refers to a functional module that may be embedded into a communication link of a biomedical system and is used for parsing, forwarding, and processing data streams. For example, the data reading plug-in includes devices such as gateways, routers, and switches. More descriptions of the biomedical systems, may be found in operation S1 and the relevant descriptions thereof.

In some embodiments, the processor pushes the newly generated merged semantic label and its corresponding data elements to the data reading plug-in through a reserved application programming interface (API). After receiving, the data reading plug-in uses the latest merged semantic label and its corresponding data elements to overwrite and update its local mapping rule library or lookup table to complete the dynamic deployment of the merged semantic label.

In some embodiments, the processor controls the data reading plug-in to perform operation S7.

S7, in response to receiving a heterogeneous service data stream containing a heterogeneous character identifier, performing a standardized payload conversion on the heterogeneous service data stream based on the merged semantic label: mapping the heterogeneous character identifier to a unified merged semantic label and appending the unified merged semantic label to a data packet; and retaining the heterogeneous character identifier as an original traceability field.

The heterogeneous service data stream refers to data originating from different biomedical systems and following different data standards or formats. A digital label of the heterogeneous service data stream itself is referred to as the heterogeneous character identifier.

The use of the heterogeneous character identifier is similar to that of the merged semantic label, both serving as the unique digital label for data. The only difference is that the merged semantic label is a globally unified standardized identifier that follows a unified generation rule and has global semantic consistency across all biomedical systems. While the heterogeneous character identifier is a non-standardized identifier, and only has a clear meaning within a single biomedical system to which it belongs. The heterogeneous character identifiers between different biomedical systems may have problems such as code redundancy, meaning conflicts, and an inability to be recognized across systems. Therefore, it is necessary to perform a standardized payload conversion on the heterogeneous character identifiers.

The standardized payload conversion is a formatting process for the heterogeneous service data stream. The standardized payload conversion only maps the heterogeneous character identifiers in the data stream to the merged semantic label, this achieves the standardization of the heterogeneous service data stream without modifying an underlying transmission protocol and without affecting normal forwarding of the data stream.

In some embodiments, the process of the standardized payload conversion is as follows. The processor sequentially traverses all the character identifiers in the heterogeneous service data stream, and determines a character identifier that is unable to be matched with standard character identifiers of a global standard terminology library as a heterogeneous character identifier, performs semantic similarity determination on the identified heterogeneous character identifier, filters out a standard character identifier with the highest similarity from the standard character identifiers of the global standard terminology library as the merged semantic label corresponding to the heterogeneous character identifier. Further, the processor maps and replaces the heterogeneous character identifier in the data stream with the matched merged semantic label, and simultaneously retains the original heterogeneous character identifier by adding a remark, as a basis for subsequent data traceability and verification.

The original traceability field is a heterogeneous character identifier specifically retained after the standardized payload conversion to maintain data source traceability.

In some embodiments, to retain the heterogeneous character identifier before mapping and to ensure that the retained heterogeneous character identifier does not interfere with character recognition of a biomedical system, the processor may retain the original traceability field in manners such as adding a remark, adding an annotation, or independently storing the original traceability as a traceability log document.

The method for automated processing of biomedical data provided by the present disclosure can utilizing the LLM for deep semantic understanding by combining the original context of the data elements within the dataset with the context fragments recalled from an external multi-source corpus, which facilitates accurately determining semantic equivalence of different data elements, helps to overcome limitations of traditional manners that rely on a single dictionary or rules. Furthermore, by generating the merged semantic label and deploying the merged semantic label to the data reading plug-in, the method achieves automated, real-time standardized conversion of data streams between heterogeneous biomedical systems while retains the original traceability field, thereby ensuring data interoperability and integrity.

The method for automated processing of biomedical data provided by the present disclosure, is not limited to the application scenarios of data unification between biomedical systems. It may also be extended to automated control scenarios in places such as hospitals, for example, pharmacy drug distribution and cross-hospital medical record merging. Hereinafter, taking a sample sorting scenario in a clinical laboratory as an example, the application scenarios of the method are further elaborated.

In the sample sorting scenario in the clinical laboratory of the hospital, sample labels generated by different campuses and disparate order entry systems lack unified standards. This leads to heterogeneous issues for the same testing item, such as inconsistent character identifiers and custom coding schemes. The control element of a traditional sorting manipulator arm only supports identifying labels that follow fixed rules, so sorting errors easily occur when it encounters heterogeneous labels, leading to high maintenance costs and low operational efficiency. By deploying the standardized merged semantic label to the control element of the sorting manipulator arm, unified recognition of heterogeneous sample identifiers can be achieved without modifying an existing sample label system and without changing underlying recognition logic of the manipulator arm. This solves pain points of sorting adaptation for medical laboratory automated assembly lines, and adapts to automated sorting demands of multi-source samples.

In some embodiments, the processor may dynamically deploy the merged semantic label to the control element of the sorting manipulator arm, and control the control element to perform a profess including: determining a target transport track for a sample to be sorted based on the merged semantic label and a character identifier of the sample to be sorted; and controlling the sorting manipulator arm to transfer the sample to be sorted to the target transport track.

The sorting manipulator arm is a sorting device that performs actions such as sample grasping, movement, and placement based on a drive signal.

The control element is a hardware unit that drives the sorting manipulator arm to perform a sorting operation. The control element may perform data communication with the processor, receive external instructions transmitted by the processor, and converts the external instructions into the drive signal readable by the sorting manipulator arm. The control element may actively provide feedback to the processor regarding parameters such as the real-time posture of the sorting manipulator arm and a sample sorting status. The sample sorting status includes not performed, in progress, completed, or abnormal alarm. The control element includes a programmable logic controller (PLC) or a microcontroller.

In some embodiments, the process for the processor dynamically deploying the merged semantic label to the control element of the sorting manipulator arm is similar to the operation in S6 where the processor dynamically deploys the merged semantic label to the data reading plug-in located between different biomedical systems, and reference may be found in above descriptions.

The sample to be sorted is a sample that has not yet been placed on the target transport track. The character identifier is an encoding attached to the sample to be sorted, used for identifying an identity or attribute of the sample to be sorted. The target transport track is a physical transport path designated for the sample to be sorted. The target transport track may transfer the sample to be sorted to its corresponding destination. The target transport track includes but is not limited to conveyor belts, guide rails, or pneumatic tube transport system.

For example, when the sample to be sorted is a blood sample of a patient, a patient barcode affixed to a body of a blood sample bottle is the character identifier, a conveyor belt for transporting the blood sample is the target transport track, and an end of the target transport track is connected to blood testing equipment.

In some embodiments, the processor may read the character identifier on the sample to be sorted through a barcode scanner or a camera. The processor sends the read character identifier to the control element. The control element queries a locally stored mapping table to find the merged semantic label corresponding to the character identifier and the target transport track corresponding to the merged semantic label. Then, the control element generates a corresponding control instruction, to drive the sorting manipulator arm to transfer the test tube sample to the target transport track. The target transport track corresponding to the merged semantic label may be determined based on prior experience.

In some embodiments, when the method for automated processing of biomedical data is applied to the sample sorting scenario, by dynamically deploying the merged semantic label to the control element of the sorting manipulator arm and performing the sorting operation, it is possible to solve the problems of high sorting error rate and high rule maintenance cost caused by heterogeneous sample identifiers without modifying the existing sample label system, and significantly improve the automated operation efficiency of the laboratory assembly line.

The application of the present disclosure is not limited to the sample sorting scenario, but may also be reused in various scenarios in the medical field. For example, by deploying the method for automated processing of biomedical data to a control terminal of an automated pharmacy, unified identification of drug information from multiple prescription systems can be achieved, which drives a drug dispensing device to accurately dispense drugs. By deploying the method for automated processing of biomedical data to a medical data center, merging and unification of medical records from different hospitals can be achieved, which facilitates cross-hospital medical visits for patients. By deploying the method for automated processing of biomedical data to the material distribution system, cross-building and cross-departmental material distribution can be achieved, the development cost for heterogeneous data adaptation in a plurality of medical scenarios can be significantly reduced, and the unification and standardization processes of business workflows in the medical industry can be promoted.

FIG. 2 is a flowchart illustrating an exemplary process for constructing a knowledge graph according to some embodiments of the present disclosure. The process may be executed by a processor. The processor may be located on any terminal device. The terminal device may include a computer, a tablet, a cell phone, or the like.

As shown in FIG. 2, some embodiments of the present disclosure disclose a knowledge graph construction approach for standard data elements of a biomedical dataset, including the following operations 110-130.

Operation 110, obtaining structured data and an unstructured text of each multivariate standard text in a multi-source standard text set.

The multi-source standard text set is a set including a plurality of multivariate standard texts. A multivariate standard text includes a data element text of a biomedical dataset and relevant standards in the biomedical field.

The data element text refers to a content text included in a data element. More descriptions of the biomedical dataset and data elements, may be found in operation S1 and related descriptions thereof.

The relevant standards in the biomedical field include, but are not limited to, dataset standards, classification and coding standards, and value domain code standards, and relevant external resources involved in biomedical datasets.

In some embodiments, the dataset standards include not only general datasets such as a directory of health information data elements, a value domain code of health information data elements, a basic dataset for disease control, a basic information dataset, a basic dataset of medical service, and a basic dataset of electronic medical record (EMR), but also standards of specialized disease datasets, such as basic datasets of electronic medical records for orthopedics, traditional Chinese medicine, hypertension, or the like. The relevant external resources may include scientific literature, medical glossaries (ICD, UMLS, etc.), etc. A level of the relevant standard of the biomedical dataset may include a national standard, an industry standard, a local standard, and a group standard.

The structured data of the multivariate standard text refers to a database composed of a set of data of specific data types, such as a medical hospital information system (HIS) database, etc. The unstructured text refers to textual content that does not have a fixed format or regularity. For example, the unstructured text includes emails, news, blogs, or the like. The structured data may be data existing in CSV, JSON, XML, and SQL formats. The unstructured text may be a text existing in PDF and DOC formats.

In some embodiments, the processor may obtain the structured data and the corresponding unstructured text of each multivariate standard text in a plurality of manners. For example, for a multi-source standard text natively published digitally (e.g., natively editable DOCX, PDF, XML files, or the like), the processor directly calls a parsing engine of a corresponding format to directly extract content such as text hierarchy, tables, and data element texts from the multi-source standard text as the structured data, and to extract content such as abstracts, text descriptions, annotations, and drawings from the multi-source standard text as the corresponding unstructured text.

In some embodiments, the processor may, by performing optical character recognition (OCR) on the multivariate standard text, to obtain the structured data and the unstructured text.

The OCR refers to a technology that scans and recognizes a text on documents and converts the recognized text into a digital text format that may be edited and processed by a computer.

In some embodiments, the processor may categorize the recognized content having a regular field structure, such as text hierarchy, tables, and data element texts, as the structured data, and categorize the recognized natural language content without fixed field rules as the corresponding unstructured text.

In some embodiments, by performing OCR to obtain the structured data and the corresponding unstructured text, the structured data and the corresponding unstructured text can be efficiently and automatically extracted from the multivariate standard text without manual document-by-document entry, thereby significantly improving the parsing accuracy and speed of the multivariate standard text.

Operation 120, extracting an entity type and attribute data from the structured data and the unstructured text.

The entity type is used to reflect the type features of the data element. In some embodiments, the entity type may include, but not limited to, 21 types including a standard, terminology, abbreviation, specified content, applicable scope, preface, introduction, sets of data elements, data element, concept of data elements, value domain code, disease, domain, department, publication, responsible institution, proposing institution, drafting institution, etc.

The attribute data refers to a characteristic that the entity type possesses. For example, the attribute data includes that a data standard and a set of data elements (also referred to as the data element set) are in an inclusion relationship, the set of data elements and the data elements are in an inclusion relationship, the set of data elements includes a plurality of data elements, or the like. For example, since the age of a patient has only one value and belongs to a scalar attribute, if the age of the patient is recorded in the unstructured text, the age of the patient is determined as the attribute data. More descriptions of the entity type and attribute data, may be found in Table 4 and related descriptions thereof.

In some embodiments, a category system and a quantity range for the entity type and the attribute data may be predefined for those skilled in the art based on experience.

In some embodiments, the processor may parse the structured data and the unstructured text based on natural language processing (NLP) to extract the entity type and the attribute data.

The NLP enables language interaction between humans and computers, as well as implements text processing, language analysis, text mining, and other tasks.

In some embodiments, the processor may perform the recognition, extraction, and annotation of the entity type and the attribute data through a field lexicon or a machine learning approach.

The field lexicon refers to a table that covers the entity type and attribute data in the biomedical field. The field lexicon includes normative expressions and synonymous expressions of different entity types and attribute data. The processor may extract commonly used entity types and attribute data from public biomedical terminologies and authoritative medical terminology sets (e.g., ICD codes, LOINC, SNOMED CT, or the like), and construct the field lexicon after organization, deduplication, and synonym merging.

In some embodiments, when the processor performs the extraction through the field lexicon, the processor matches the structured data and the unstructured text with all expression items of the entity type and the attribute data stored in the field lexicon one by one. Upon successful matching, the entity type and the attribute data from the field lexicon that are matched are determined as the extracted entity type and attribute data.

In some embodiments, the machine learning approach includes using an NLP model to perform recognition, extraction, and annotation.

In some embodiments, the processor may perform natural language processing through a NLP model, such as BioBERT, ClinicalBERT, BlueBERT, or the like, that are suitable for the biomedical field. The processor may use the structured data, the unstructured text, the preset 21 types of entity types, and the attribute data as the input for the NLP model, and provide instructions to the NLP model in natural language to guide the NLP model to match the most appropriate entity type and attribute data for the input structured data and unstructured text. The processor determines the output result of the NLP model as the semantic consistency score.

In some embodiments, based on the field lexicon, known standardized entities and attributes can be quickly matched, thereby greatly improving extraction efficiency in routine scenarios. According to the machine learning approach, it may cover emerging terms and non-standard expression scenarios not included in the field lexicon, thereby effectively expanding the extraction range. The two approaches complement each other, which not only ensures the professional accuracy of the entity and attribute extraction in the biomedical field but also enhances the adaptability to data sources with different formats and expression habits.

In some embodiments, based on NLP, the structured data and the unstructured text are parsed, and the entity type and the attribute data are extracted. NLP can quickly and accurately parse the structured data and the unstructured text. Compared to traditional rule matching and manual annotation extraction manners, it can significantly improve the data processing speed, and is especially suitable for multi-source, heterogeneous multivariate standard texts with inconsistent expressions.

In some embodiments, the operations of extracting the entity type and attribute data from the structured data include:
identifying and extracting content of the relevant standard texts of the data elements using a human-machine collaboration manner; performing data cleaning, data review, and data quality control on the extracted content, writing a regular expression of identifier data according to a clearly defined coding rule, performing spelling check and quality control on different codes, correcting a problematic identifier, and unifying identifiers; if the extracted content includes a recognition error, useless space or line break, or a garbled code or omission, supplementing and modifying, by human beings, the extracted content to complete extraction and organization of the content of the relevant standard texts and form preliminary structured data.

The standard documents referenced in the present disclosure, whether dataset standards, subject code standards, or code value domain standards, all have different text structures. By referring to guidelines such as WS/T370-2022 Compilation Standard of Basic Health Information Dataset and T/CHIA6-2018 Specification for the Compilation of Specialized Electronic Medical Record Dataset, and considering differences between actual texts and national, industry, regional, and group standards, each type of text structure may undergo text analysis and content unit identification. Common content units across various types of standards are merged, and common features are extracted, while unique units are extracted separately. For different text structures, a database is designed for storing an extracted structured object.

Since a type of a text belongs to the unstructured text, which is mostly in .pdf or .doc formats, the recognition and extraction of text content may be carried out through the human-machine collaboration manner. The machine manner may mainly include the OCR image recognition and PDF content extraction techniques. For example, extracted text content includes a preface, introduction, prescribed content, scope of application, cited documents, terminology, acronyms, references, and so on. The extracted text content may be subjected to data cleaning, data review, and data quality control.

In some embodiments, the processor may identify and extract the data element text of the multivariate standard text; perform data cleansing, review, and quality control on the data element text; write a regular expression according to a coding rule to perform identifier spell check and unified correction on the data element text; and supplement and modify problems existing in the data element text, including a recognition error, a garbled text, or an omission, to form preliminary structured data.

In some embodiments, because the text quality of data element texts from different sources differs, if a data element text with problems such as recognition errors, garbled text, omissions or non-standard formats is directly used for the extraction of the entity type and attribute data, it will ultimately affect the accuracy and reference value of the finally generated knowledge graph. Therefore, it is necessary to perform the data cleansing, review, and quality control on the data element text.

In some embodiments, the data cleansing may include: the processor traversing all data element texts to clean meaningless spaces, line breaks, and special characters; automatically identifying and replacing approximate characters confused by OCR recognition through a preset recognition error mapping table; and identifying and replacing non-standard symbols such as double periods and single-sided parentheses, and errors such as repeated words and misspelled characters, in the data element text.

In some embodiments, the processor may perform the review and the quality control based on the aforementioned NLP model. The processor uses the data-cleansed data element text as input to the NLP model, provides instructions to the NLP model to review the input text, and outputs a review result. If the review result indicates that the data-cleansed data element text is still unqualified, the processor triggers a secondary cleansing process. The processor re-examines and re-modifies the data element text and submits the data element text for review again to complete the quality control of the data element text.

The coding rule is a rule requirement that the data element text must satisfy. For example, for identifier data such as a standard number, an internal identifier, etc., a regular expression of the identifier data is written according to a clearly defined coding rule, spelling check and quality control on different codes are performed, a problematic identifier is corrected, and identifiers are unified, so as to facilitate normalization and statistics. The standard number is unique and may be used directly for the construction of the knowledge graph of data elements. However, identifiers of data elements may be duplicated in different standards, they cannot be used directly for identification, and need to be redefined as unique codes.

Additionally, extracted content may include recognition errors, unnecessary spaces, line breaks, garbled text, omissions, etc., which require manual supplementation and modification to complete the extraction and organization of entire text content, so as to form the preliminary structured data. The regular expression refers to a text pattern that describes a rule for string matching. The regular expression can facilitate retrieval, replacement, validation, and other operations on data elements.

In some embodiments, by performing the data cleansing, the review, and the quality control on the data element text, and ultimately forming the preliminary structured data, the quality of the structured data can be effectively improved, ensuring that all the structured data satisfies quality standards.

In some embodiments, the operation of extracting the entity type and the attribute data from the unstructured text in the structured data includes:

manually annotating and performing review and quality control on the entity type by recognizing, extracting, and annotating from the unstructured text in the structured data using a field vocabulary or machine learning manner.

Not all entity types originate from the structured data, and data that characterize features of biomedical field standards may need to be extracted and annotated from unstructured descriptions (e.g., titles, abstracts, etc.) in the structured data using field vocabularies or machine learning manners. The entity types (e.g., diseases, departments, subject headings, etc.) need to be manually annotated and subjected to quality control, so as to enrich and enhance a field feature and an application scenarios feature of the data element standard and the data element, and then realize more fine-grained and multi-dimensional content revelation ranging from biomedical field standards to sets of data elements, data elements to value domains, and so on.

The concept identification of data elements: among dataset standards collected in the present disclosure, a small number of dataset standards (for example, a group standard issued by the Guangdong Provincial Hospital Association) cover specialized fields such as chronic diseases, hypertension, coronary heart disease, and cerebral infarction. The standard references a standard of ISO/IEC 11179 Metadata Registration System. For example, in a group standard such as T/GDPHA 031-2021 General Standard Dataset for Cerebrovascular Disease Research, mappings between data elements and concepts of data elements in vocabularies or common data element repositories such as CDISC, SNOMED CT, LOINC, and NIH CDE have been implemented, with concept English names or concept ID codes annotated. Therefore, a relationship between data elements and corresponding concepts of data elements may be extracted from such dataset standards.

The extraction of concepts of these data elements is based on English vocabularies/ontologies of medical field. However, most data elements in dataset standards do not define concepts of data elements and are expressed in Chinese. Therefore, the present disclosure uses Chinese/English vocabularies/ontologies of medical field to obtain the concepts of data elements. The subject vocabulary of medical field includes subject terms and entry terms, and has a hierarchical structure of concepts. Each subject term contains multiple entry terms with synonymous relationships. By matching data elements with subject terms and the entry terms under each subject term, the concept of the data element may be obtained.

In addition, if specific resources are involved, data must be extracted from texts or supplemented with external link information to ensure data association and resource accessibility. Specific resources may include reference papers, cited policies, cited standards, and other resources.

The knowledge graph refers to a data structure that organizes standard data elements in a form of nodes and relationships. The knowledge graph can help users better understand and work with complex standard data elements.

In some embodiments, the processor may analyze and summarize raw data involved in the relevant standard texts regarding provisions in the data of the relevant standards of the biomedical dataset, so as to obtain analyzed and summarized data. For example, if the data of the relevant standard of the biomedical dataset includes Compilation Standard of Basic Health Information Dataset, the processor may, according to the Compilation Standard of Basic Health Information Dataset, analyze and summarize raw data from relevant standard documents of the data elements to obtain analyzed and summarized data.

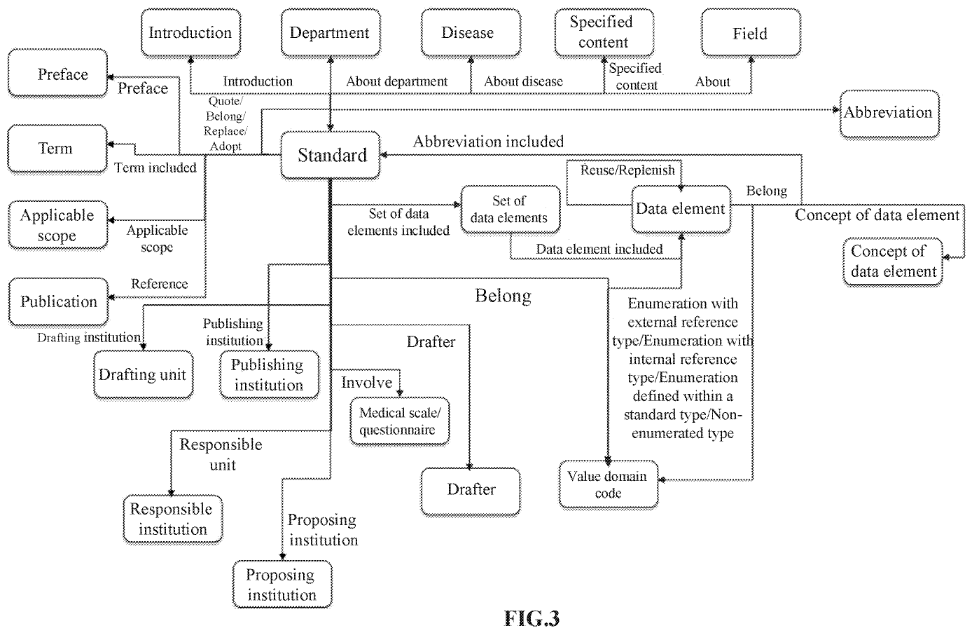

FIG. 3 is a schematic diagram illustrating a knowledge model according to some embodiments of the present disclosure.

In some embodiments, the knowledge model may be a knowledge model as shown in FIG. 3.

Operation 130, performing knowledge fusion on the entity type and the attribute data based on the semantic associative relationship to generate the knowledge graph.

In some embodiments, the semantic associative relationship include: a relationship between data standards, a relationship between a set of data elements and the data elements, a relationship between the data elements and concepts of the data elements, a relationship between the date elements, a relationship between the data elements and value domains of the data elements, a relationship between a dataset standard and a medical scale/questionnaire, and a relationship between the data elements and the medical scale/questionnaire.

In some embodiments, the inter-standard data relationship is multiple relationships. The relationship between the data standard and the data element set is a containment relationship. The relationship between the data element set and the data element is a containment relationship, wherein a plurality of the data elements are included under the data element set. The relationship between the data element and the data element concept is a mapping relationship, wherein the data element corresponds to a unique data element concept. The relationship between the data element and the value domain includes: an enumerated reference type, a self-enumerated reference type, an enumerated definition type, or a non-enumerated type. The relationship between data elements includes: a synonymous relationship, a relevant relationship, or an irrelevant relationship. The relationship between the dataset standard and the medical scale or the questionnaire is the reference relationship. The relationship between the data element and the medical scale or the questionnaire is the reference relationship.

In some embodiments, the semantic associative relationship may be presented in the form of a knowledge model. For more information about the knowledge model, please refer to Table 2, Table 3, and related content below. For more information about determining the semantic associative relationship, please refer to operations I-VII and related descriptions below.

The knowledge fusion refers to a process for integrating information of a same data element in biomedical datasets from different data sources to obtain more comprehensive information about the data element. More content about how to perform the knowledge fusion on the plurality of types of data can be referred to operations c1-c4 below.

In some embodiments, the knowledge graph construction approach further includes: storage and quality checking of the knowledge graph. Storage includes: constructing an entity attribute table and a triple relationship table, batch converting data into Unicode Transformation Format-8-bit encoding (UTF-8 encoding), and importing the data into a graph database for storage. The quality checking includes: after importing the data, verifying correctness of triple data through sampling to ensure accuracy of the entity type and an associated relationship.

The entity attribute table refers to a table used to represent a correspondence between an entity type and a corresponding attribute.

The tripe relationship table may be configured to represent semantic relationships between different entities. The triple relationship table may be a basic building unit of the knowledge graph.

The triple data refers to a data structure including three elements. The three elements may include a subject, a predicate, and an object.

In some embodiments, a person skilled in the art may predefine the plurality of entity attribute tables and the plurality of entity triple relationship tables based on experience. For more information about the triple relationship table, please refer to Table 7 below.

UTF-8 refers to a variable-length character encoding manner for representing all characters in the Unicode character set.

The Neo4j graph database may be understood as a high-performance NoSQL graph database including the triple data.

In some embodiments, storage of the knowledge graph includes:

Establishing a plurality of entity attribute tables and a plurality of entity triple relationship tables, performing batch conversion, importing, and converting triple data (e.g., subject, predicate, object) to UTF-8 to avoid encoding issues; and storing the knowledge graph with a Neo4j graph database. For importing data into the Neo4j graph database, a Neo4j-import tool may be used to import organized structured triple data to form a final knowledge graph. All data may then be queried and visualized through Cypher queries, supporting the query of a relationship between entity types in the knowledge graph of the standard data element of the biomedical dataset.

In some embodiments, storing and checking the knowledge graph helps to ensure the storage compatibility, data integrity, and relationship accuracy of the knowledge graph. Adopting a unified UTF-8 encoding can avoid issues such as garbled text and character recognition errors during storage. By constructing the entity attribute table and the triple relationship table, the attribute data of the entities themselves is clearly preserved, and the semantic associative relationships between the entities are completely recorded, which adapt to the storage and query logic of the graph database. The sampling verification stage after import can identify issues such as annotation errors of the entity types and deviations in associative relationship matching, thereby ensuring the data quality of the knowledge graph.

In some embodiments, the process for constructing types of semantic associative relationships between one or more entity types is as follows.

The following highlights a process for defining and processing relationships between a plurality of important entity types that need to be constructed.

I. A relationship between data standards is a multiple relationship. The relationship between data standards is pluralistic. For example, a dataset standard references other standards, a new standard replaces a deprecated standard, a standard follows other standards, and so on. Additionally, a compositional relationship between standards is often overlooked. A biomedical dataset standard may include a plurality of standards. For example, an electronic medical record dataset for hypertension specialties includes 14 sections. Standards in these sections together form a dataset standard, and a relationship between these standards is that they are components of a same dataset. A value domain standard is similar. For example, WS 364 Health Information Data Element Value Domain Code includes 17 sections, such as demographic and socio-economic characteristics, health history, health risk factors, etc. Among the sections, except for the first and second sections, which are compilation rules, the remaining 15 sections are available code tables. The 15 sections together form a value domain of health information data elements. A relationship between data standards is shown in a table below:

TABLE 1
Relationship between data standards

| Entity type 1 | Relationship type | Entity type 2 |
|---|---|---|
| Dataset standard | Belong | Dataset standard |
| Dataset standards | Quote | Standard |
| Dataset standard (changes and | Replace | Dataset standard |
| modifications) | | |
| Dataset standard | Quote | Publications |
| Set of data elements | Replenish | Data elements |
| Set of data elements | Reuse | Data elements |
| Set of data elements | Include | Data elements |
| Dataset standard | Refer | Publications |
| Dataset standard | Include | Value domain |
| Dataset standard | Involve | Medical |
| | | scales/questionnaires |

II. A relationship between a set of data elements and data elements is a containment relationship. The set of data elements may be specifically reflected in a biomedical dataset standard as a set of specialized attributes for data elements with specific names. Each set of specialized attributes for data elements may typically include a plurality of data elements. The division of sets of data elements is ignored in existing research and applications. In the dataset standard, a specialized attribute of the data element includes the classification of the data element. For example, General Data Element Standards for Clinical Scientific Research on Gastric Cancer includes sets of seven specialized attributes of data elements. The sets of seven specialized attributes of data elements may include sets of: a general data element, demographic basic information of a subject, an outpatient (emergency) medical record of a subject, examination information of a subject, test information of a subject, admission and discharge information of a gastric subject, and adverse event information of a subject. Therefore, the data standard and the set of data elements may be in an inclusion relationship, and the set of data elements and the data elements may be in an inclusion relationship, the set of data elements may include a plurality of data elements.

III. A relationship between data elements and concepts of data elements. Data elements may mainly originate from specific attributes of data elements, which most of the data elements in a standard of Chinese biomedical dataset do not provide concepts of data elements and information of subjects as required by a standard of ISO/IEC 11179 “Metadata Registration System”. This part needs to be supplemented with the concepts of data elements using a subject vocabulary of medical field, or the like.

IV. A relationship between data elements. The relationship between the data elements may include three types including a synonymous relationship, a relevant relationship, and an irrelevant relationship. Specifically, the relationship between the data elements is realized through following IV-1 to IV-6:
IV-1. After identifying the concepts of the data elements, performing synonymous relationship recognition on the data elements, and if the concepts of two data elements in the data elements are the same in each subject vocabulary of a same medical field, determining the two data elements being in the synonymous relationship, and marking a similarity between the two data elements as 1;
IV-2. If the two data elements are in a non-synonymous relationship, performing similarity calculation on the two data elements with completely different standard codes and data element identifiers using a Jaccard similarity manner by determining a ratio of an intersection set to a concatenation set between sets corresponding to the two data elements, respectively, wherein a calculation formula is shown in the following Formula:

$$Sim\_ele ( E 1 , E 2 ) = \frac{\vert A ∩ B \vert}{\vert A \vert + \vert B \vert - \vert A ∩ B \vert}$$

where, E1 and E2 denote two data elements, respectively, a tokenization processing is performed on a text of each of the two data elements, E denotes a tokenized text composed of a data element name and a data element definition of the two data elements, Sim_ele_name( ) denotes a data element similarity, A denotes a tokenized text of E1, and B denotes a tokenized text of E2, and a final similarity result is controlled to be in a range of [0, 1]. The tokenized text refers to a separate lexical unit cut from a continuous text.
IV-3. If the two data elements are in the non-synonymous relationship, calculating a similarity between a first data element and a second data element in the two data elements according to the calculation formula; if the similarity between the two data element s is greater than a data element synonymity threshold, determining the first data element and the second data elements being in a candidate synonymity relationship;
IV-4. If the similarity between the two data elements is greater than a data element relevance threshold and less than the data element synonymity threshold, then determining the first data element and the second data element are being in a candidate relevance relationship;
IV-5. If the similarity between the data elements is less than the data element relevance threshold, recording the similarity between the two data elements only, and marking a relationship between the first data element and the second data element as irrelevant;
IV-6. A candidate relationship between each pair of data elements may not be obtained through similarity calculation alone, but also through manual verification and adjustment to determine an exact relationship to ensure the accuracy of the relationship. This establishes the multi-dimensional fine-grained correlation and degree of association between data elements, providing intelligent recommendations for subsequent creation and reuse of data elements.

V. A relationship between data elements and value domains of the data elements. The value domain of the data element refers to a value range of the data element, e.g., a range of blood pressure. The present disclosure refines the relationship between the data element and the value domain of the data element, and divides a usage manner of the value domain of the data element at a fine-grained level. The value domain of the data element may be classified into four types including an enumerated reference type, a self-enumerated reference type, an enumerated definition type, and a non-enumerated type based on a source of the value domain and the usage manner.

The enumerated reference type refers to referencing a value domain table of the other standard (different from the standard where the data element and the value domain are located), with a clear value domain standard or a table name provided.

The self-enumerated reference type refers to referencing a value domain table defined within the same standard where data element and value domain are located, entries of allowed values being more than 4, with clearly specified table name and table code.

The enumerated definition type refers to that within a standard where the data element and the value domain are located, the allowed values are defined directly in the data element section without using a form of value domain table. Typically, a count of entries of the allowed values is fewer than 4.

The non-enumerated type refers to a value domain that is not listed by entries of the allowed values. The non-enumerated type may be typically identified using text to describe the allowed values or free-fill.

Based on the above definitions and methodology, a process for determining a relationship between the data elements and the value domains of the data elements may include the following operations V-1 to V-4:

Operation V-1, determining, based on the data element and a value domain corresponding to the data element, whether an allowed value of the data element includes a standard number or a number or name of a value domain code table, judging through a coding rule base, and in response to determining the allowed value including the standard number or the number or name of the value domain code table, determining the value domain of the data element as enumeration reference; or in response to determining the allowed value not including the standard number or the number or name of the value domain code table, performing Operation V-2.

Operation V-2, if the value domain of the data element is enumeration reference, further judging if a standard number of a dataset of the value domain is the standard number of the data element or if a number of a value domain code table of the value domain is the number of the value domain code table of the data element, and in response to determining the standard number of the dataset being different from the standard number of the data element or the number of the value domain code table of the value domain not including the number of the value domain code table of the data element, determining the value domain of the data element as the enumerated reference type; and in response to determining the standard number of the dataset being the same as the standard number of the data element or the number of the value domain code table of the value domain including the number of the value domain table of the data element, determining the value domain of the data element as the self-enumerated reference type.

Operation V-3, in response to determining the allowed value of the data element not including the standard number or the number or name of the value domain code table and the allowed value includes “;” determining a split numeric item as the enumerated definition type.

Operation V-4, in response to determining the split numeric item not belonging to the enumerated definition type, determining the split numeric item as the non-enumerated type.

VI. A relationship between a dataset standard and a medical scale/questionnaire. The relationship between a dataset standard and a medical scale or a questionnaire is a reference relationship. The medical scale may be used in the dataset standard, and a scale name and information of the medical scale may be extracted from a text, and a connection between a specific medical scale and data element is established by complementing resources of the medical scale.

VII. A relationship between a data element and a medical scale/questionnaire.

The relationship between a data element and a medical scale or a questionnaire is a reference relationship. In some embodiments, a relationship between the dataset standard, the set of data elements, the data element, and the value domain code is shown in FIG. 5. FIG. 5 is a schematic diagram illustrating relationships between dataset standards, sets of data elements, data elements, and value domain codes according to some embodiments of the present disclosure.

In some embodiments, knowledge fusion of a plurality of types of data includes operations c1-c4 as follows.

In some embodiments, a processor may achieve the knowledge fusion through knowledge merging, entity disambiguation, co-reference resolution, or the like. Example data of different entity types need to be de-duplicated and disambiguated, and targeted processing is performed based on the characteristics of different entity types.

Operation c1, disambiguating using a pre-existing unique code, including processing a cross-level number. For example, in the data standard, even though a standard number is unique, different descriptions of a same standard (such as standard name, standard number, and abbreviation) may appear in different locations within a dataset standard document, which may lead to a same object not being recognized consistently.

Similarly, there are differences in a name of value domain code table, and a name of a vocabulary. For example, CV03.00.107, WS364.5CV03.00.107 Dietary Habits Code Table, and Part 5 of WS364.5 Health Information Data Element Value Domain Code actually all correspond to a same value domain code table. There is also an issue of code duplication between internal and external codes, as there is currently no fine-grained Chinese data element query system available, which leads to the occurrence of code duplication.

Operation c2, standardizing a name. Normalization and merging of a name is required because there are different expressions for a same organization name. For example, if Health Department Statistics Information Center, Ministry of Health Statistics Information Center of the People's Republic of China, and Health Ministry Health Statistics Information Center all refer to a same entity, then standardization and merging of the names are required. Therefore, the normalization of naming and coding is implemented through regulations and standards including WS/T306 Rules for Classification and Coding of Health Information Datasets and WS370-2012 Rules for Formulating Specifications for the Preparation of Basic Health Information Datasets, an institutional specification library and a field vocabulary, similarity calculation, manual verification, and quality control.

Terms, acronyms, etc., are also semantically merged through a field subject vocabulary, a general subject vocabulary, and so on. The field subject vocabulary refers to a systematic vocabulary list that is organized, classified, compiled, and arranged to cover all the subjects within a specific academic field. The general subject vocabulary may be a list of universally used field-specific terms.

Operation c3, merging names of the data elements through similarity calculation between the data elements, merging of the concepts of the data elements, and manual discrimination.

Operation c4, merging names of data value domain tables. The value domain table in a standard text of a dataset and an allowed value of the data element may be both related to a relevant name of the data value domain table including a table number, a table code, and a table name. It is required to perform structured processing on the table number, the table code, and the table name, correct, combine, and merge the table number, the table code, and the table name, and fuse a standard number to realize merging and disambiguation of the names of data value domain tables. The relevant name of the data value domain table may include a table number, a table code, and a table name. The standard number may be interpreted as a corresponding unique representer of an associated name of a data value domain table.

In some embodiments, data update includes the following content.

With the establishment of new standards for biomedical dataset and the revision of existing standards, content will change accordingly. Collection and processing of data related to dataset standards and data elements are ongoing, and updating and supplementing of example data corresponding to entity types for changed content are performed. The data elements, dataset standards, and institutions may be merged and a newly generated type of semantic associative relationship and data may be supplemented into the knowledge graph of the standard data element of the biomedical dataset.

Merely by way of example, a specific embodiment is introduced to further explain the method for automated processing of biomedical data provided by the present disclosure.

(1) Designing an entity type and a relationship between entity types of a knowledge model, as shown in Table 2 and Table 3.

TABLE 2
Examples of entity types

| Serial | | |
|---|---|---|
| number | Entity type | Illustration |
| 1 | Data standard | A standard of for standardizing a dataset, value domain code, |
| | | classification code, etc. |
| 2 | Term | A term used in a standard |
| 3 | Abbreviation | An abbreviation used in a standard |
| 4 | Stipulated | Main technical content of standard content |
| | content | |
| 5 | Scope of | A scope of application, scenario, etc. of a standard |
| | application | |
| 6 | Preface | A corresponding preface section in a standard text |
| 7 | Introduction | A corresponding introductory section in a standard text, which |
| | | is not included in all standard texts |
| 8 | Domain | A secondary classification corresponding to a subject |
| | | classification |
| 9 | Publication | A reference of a standard text |
| 10 | Unit | A hospital department that may correspond to a certain disease |
| | | based on content of s standard |
| 11 | Disease | About a disease for which an ICD10 code is desirable, based on |
| | | content of a standard |
| 12 | Data element | A data element in a standard text |
| 13 | Concept of | A normalized name and source of a data element |
| | data element | |
| 14 | Responsible | A standardized name of a responsible institution |
| | institution | |
| 15 | Proposing | A standardized name of a proposing institution |
| | institution | |
| 16 | Drafting | A standardized name of a drafting institution |
| | institution | |
| 17 | Registering | A standardized Name of a registering institution |
| | institution | |
| 18 | Set of data | A categorized set of a plurality of data elements based on a |
| | elements | feature of data elements |
| 19 | Value domain | A range, source, etc. of a value of a data element |
| | code | |
| 20 | Drafter | A standardized name of a drafter |
| 21 | Medical scale | A medical scale is a standardized measurement tool consisting |
| | | of multiple items designed to reveal the level of theoretical |
| | | variables that are not amenable to direct methods of |
| | | measurement |

TABLE 3
Examples of relationship between entity types

| Serial | | | |
|---|---|---|---|
| number | Entity type 1 | Attributes of relationships | Entity type 2 |
| 1 | Standard | Quote | Standard |
| 2 | Standard | Belong | Standard |
| 3 | Standard | Adopt | Standard |
| 4 | Standard | Replace | Standard |
| 5 | Standard | Preface | Preface |
| 6 | Standard | Introduction | Introduction |
| 7 | Standard | Includes terminology | Terminology |
| 8 | Standard | Includes abbreviations | Abbreviation |
| 9 | Standard | Scope of application | Scope of application |
| 10 | Standard | Stipulated content | Stipulated content |
| 11 | Standard | Refer | Publications |
| 12 | Standard | Related units | Unit |
| 13 | Standard | Related Diseases | Disease |
| 14 | Standard | Relevant domains | Domain |
| 15 | Standard | Draft | Drafting institution |
| 16 | Standard | Publish | Publishing institution |
| 17 | Standard | Response | Responsible institution |
| 18 | Standard | Propose | Proposing institution |
| 19 | Standard | Draft | Drafter |
| 20 | Standard | Contains a set of data elements | Set of data elements |
| 21 | Set of data | Include | Data elements |
| | elements | | |
| 22 | Data element | Belong | Concept of the data |
| | | | element |
| 23 | Data element | Enumeration with external | Value domain code |
| | | reference | |
| 24 | Data element | Enumeration with internal | Value domain code |
| | | reference | |
| 25 | Data element | Enumeration defined within a | Value domain code |
| | | standard | |
| 26 | Data element | Non-enumerated | Value domain code |
| 27 | Data element | Reuse | Data element |
| 28 | Data element | Replenish | Data element |
| 29 | Standard | Include | Value domain code |

(2) An example of extracting structured entity types from structured data and unstructured data as shown in Table 4, Table 5 and Table 6.

TABLE 4
Extracted structured entity types and attributes-Standard example

| | |
|---|---|
| Standard number | WS 375.20-2016 |
| International | 11.020 |
| Standard | |
| Classification | |
| Number | |
| China Standard | C 07 |
| Classification | |
| Number | |
| Standard Chinese | [symbol] 20 [symbol] : [symbol] |
| name | |
| Standard type | Dataset standard |
| Standard English | Basic dataset of discase control-Part 20: Registration and report of |
| name | stroke |
| Catalog | Industry standard |
| Publisher | National Health and Family Planning Commission of the People's |
| | Republic of China |
| Preface | WS 375, Basic dataset of discase control, is now divided into the |
| | following parts: |
| | Part 1: Comprehensive AIDS Control; . . . |
| | This part is Part 20 of WS 375. This part is drafted in accordance with |
| | the rules given in GB/T 1.1-2009. |
| | Main drafting unit of this part: Shanghai Center for Disease Control and |
| | Prevention, Shanghai Institute of Preventive Medicine, Shanghai |
| | Municipal Health and Family Planning Commission, Shanghai |
| | Municipal Health and Family Planning Commission Information Center. |
| | Principal drafters of this part: Wu Fan, Miao Falcon, Li Xinjian, Zhong |
| | Weijian, Xia, Jiang Zhihai, Cai Chun, Xie Hua. |
| Drafting Institution | Shanghai Center for Disease Control and Prevention, Shanghai Institute |
| | of Preventive Medicine, Shanghai Municipal Health and Family |
| | Planning Commission, Information Center of Shanghai Municipal |
| | Health and Family Planning Commission |
| Stipulated content | WS 375 specifies attributes of metadata of datasets and attributes of data |
| | elements of a basic dataset of the registration and report of stroke. |
| Scope of | This part applies to disease prevention and control organizations, |
| application | medical institutions providing related services and health administrative |
| | departments for related business data collection, transmission and |
| | storage. |
| Disease | Stroke (ICD10/163.9) |
| Unit | neurology |
| Referenced | GB/T 2260 Administrative division codes of the People's Republic of |
| standard | China |
| | GB/T 2261.1 Classification and Coding of Personal Basic Information- |
| | Part 1; Sex Codes for Persons |
| | GB/T 2261.2 Classification and Codes of Basic Personal Information- |
| | Part 2: Marital Status Codes |
| | GB/T 3304 Roman Alphabet Spelling and Codes for the Names of |
| | China's Ethnic Groups |
| | GB/T 4658 Academic Calendar Codes |
| | GB/T 6565 Occupations Classification and Codes |
| | WS 364.3 Value domain codes of health information data elements-Part |
| | 3: Demographic and socio-economic characteristics |
| | WS 364.5 Value domain codes of health information data elements-Part |
| | 5: Health risk factors |
| | WS 364.11 Value domain codes of health information data elements- |
| | Part 11: Medical assessment |
| | WS 370 Specifications for the Preparation of Basic Health Information |
| | Datasets |
| | ICD-10 International Standard Classification of Diseases codes |

TABLE 5
Extracted structured entity types and attributes-Example of data elements

| | | | | Identi- | | |
|---|---|---|---|---|---|---|
| | | Chinese | | fication | Data | Allowable |
| | | name | | format | type | value of |
| External | Internal | of data | Definition of | of data | of data | data |
| identifier | identifier | element | data element | element | element | element |
| HDSD00. | DE05.01. | [symbol] | Codes for TCM | S3 | N1 | T/CIATCM |
| C6.003 | 501.00 | [symbol] | Clinical | | | 003-2019 |
| | | [symbol] | diagnostic | | | Chinese |
| | | | methods in | | | Medicine |
| | | | specific coding | | | Information |
| | | | systems | | | Data |
| | | | | | | Elements |
| | | | | | | Value |
| | | | | | | Domain |
| | | | | | | Code |
| | | | | | | 05.01.501 |
| | | | | | | Chinese |
| | | | | | | Medicine |
| | | | | | | Diagnosis |
| | | | | | | Code Table |
| HDSD00. | DE05.01. | [symbol] | Codes for TCM | S3 | N1 | T/CIATCM |
| C6.004 | 502.00 | [symbol] | clinicians use | | | 003-2019 |
| | | | vision to observe | | | Chinese |
| | | | abnormal | | | Medicine |
| | | | changes in the | | | Observation |
| | | | patient’s spirit, | | | Code List of |
| | | | color, shape, | | | Traditional |
| | | | form, tongue, | | | Chinese |
| | | | excretions, and | | | Medicine |
| | | | pediatric | | | Information |
| | | | fingerprints in | | | Data |
| | | | order to | | | Elements |
| | | | understand the | | | Value |
| | | | diagnostic | | | Domain |
| | | | content of the | | | Code |
| | | | condition in | | | |
| | | | specific coding | | | |
| | | | systems | | | |

TABLE 6
Structured data elements and concepts of data elements

| | | | | | SNOMEDCT |
|---|---|---|---|---|---|
| | | Identifier of | Name of | CDISC concepts | concepts of |
| Standard | Internal | data | data | of data | data |
| number | identifier | element | element | elements | elements |
| T/GDPHA014- | LV010101.001. | DE02.01.00 | lymphatic | \ | C96660 |
| 2021 | 04.020 | 4.01 | node | | |
| | | | transfer | | |
| T/GDPHA013- | COPD010101.0 | DE02.01.00 | exertion | Forcevitalcapac- | C111361 |
| 2021 | 01.04.030 | 4.11 | spirometry | itymeasurement | |
| T/GDPHA013- | COPD010101. | DE02.01.00 | Exertion | FVCVolRespira- | |
| 2021 | 001.04.031 | 4.12 | spirometry | toryPredicted | |
| | | | predictions | | |

(3) Based on an association relationship between entity types constructed by the knowledge graph, generating a triple group, as shown in Table 7 and Table 8.

TABLE 7
Triple data of entity type

| SetID: | | |
|---|---|---|
| ID | Name | :LABEL |
| SetID1 | Specialized Attributes of Common Data Elements for Chronic | Set of data |
| | Disease Clinical Research Program Information | elements |
| SetID2 | Specialized Attributes of a subset of data elements for basic | Set of data |
| | demographic information of Subjects for Chronic Disease | elements |
| | Clinical Research | |
| SetID3 | Specialized Attributes of Data Elements of Outpatient | Set of |
| | (Emergency) Clinic Records of Subjects for Chronic Disease | data elements |
| | Clinical Research | |
| SetID4 | Specialized Attributes of Data Elements of Inspection | Set of data |
| | information of Subjects for Chronic Disease Clinical Research | elements |
| SetID5 | Specialized Attributes of Data Elements of Examination | Set of data |
| | information of Subjects for Chronic Disease Clinical Research | elements |

TABLE 8
Data of references of entity type standards

| StID: START_ID | StID: END_ID | :TYPE |
|---|---|---|
| T/GDPHA 002-2021 | GB/T 2261.1-2003 | Quote |
| T/GDPHA 002-2021 | GB/T 2261.2-2003 | Quote |
| T/GDPHA 002-2021 | GB/T 2261.4-2003 | Quote |
| T/GDPHA 002-2021 | GB/T 3304-1991 | Quote |

(4) Performing fusion and construction on a knowledge graph to achieve merging of entities through rules and dictionaries.

For example, CV03.00.107, WS364.5 CV03.00.107 Dietary Habits Code Table, WS364.5 Health Information Data Elements Value Domain Codes-Part 5, may be unified and merged into WS364.5 CV03.00.107 Dietary Habits Code Table.

As another example, the Statistical Information Center of the Ministry of Health, the Statistical Information Center of the Ministry of Health of the People's Republic of China, the Health Statistics Information Center of the Ministry of Health of the People's Republic of China may be merged into the Statistical Information Center of the Ministry of Health of the People's Republic of China.

(5) Storing data and performing quality inspection.

After importing all the triple data into the Neo4j graph database, a data sampling check is performed to verify the correctness of the triple data and to ensure that the entity types and association relationships are correct.

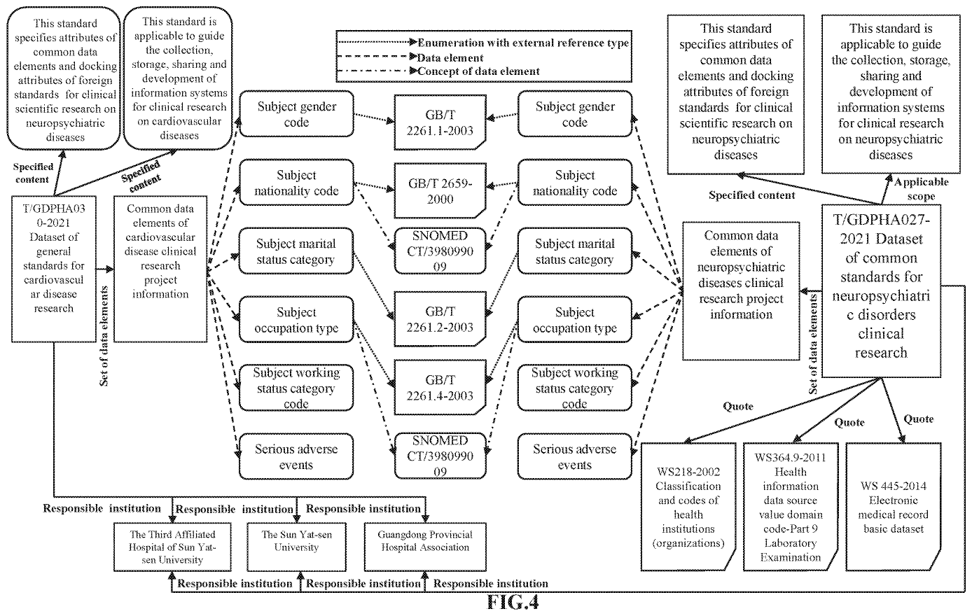

FIG. 4 is a schematic diagram illustrating a data instance of a portion of a knowledge graph according to some embodiments of the present disclosure. The data instance of a portion of the knowledge graph finally established is as shown in FIG. 4.

The present disclosure provides a method for automated processing of biomedical data, in an aim to collect dataset standards, classifications of data standards, and value domain standards in the field of biomedical science data, performing fragmented and standardized processing, and merging semantic meanings of data elements through part-of-speech and semantic calculations to establish effective associations. Subsequently, a knowledge model of the standard data element of the biomedical dataset is designed and the knowledge graph is constructed to support the standardization of data fields/variables and their value domains. The present disclosure takes the standard data element of the biomedical dataset as an example, and the method and system disclosed can be generalized to the design and implementation of knowledge graphs of data elements of datasets in other fields. On one hand, the method and system disclosed can enhance the field-specific sets of data elements, classification of data elements, and usability and utilization of value domain standards. On the other hand, it is conducive to achieving the unification of data elements and the standardization of establishment of the sets of data elements, refinement, and enrichment of the association between different dataset standards, sets of data elements, data elements, concepts of data elements, and value domains of data elements, thereby improving the machine readability and semantic interoperability.

The present disclosure analyzes and summarizes structures of standards at different levels and fields and features of standard data elements of important datasets to support to construct the knowledge model of the knowledge graph of the standard data element of the biomedical dataset, parse the data, and extract the fine-grained content.

In some embodiments, a core of the construction of the knowledge graph lies in the design and construction of a graph knowledge model oriented to specific needs. Although a small portion of existing studies focus on the construction of knowledge graphs for general standard texts, issues such as coarse knowledge granularity, low standardization, and weak association are common. There is a lack of fine-grained framework modeling, knowledge extraction, and establishment of association specifically tailored to particular fields and applications. Additionally, the construction of machine-readable dataset standards, reuse of data element and value domains remain insufficient. During data processing and construction of graphs, the present disclosure mainly refers to a standard of ISO/IEC 11179 Metadata Registration System, Compilation Standard of Basic Health Information Dataset, and other relevant guidelines and is aimed to satisfy business needs and development goals such as the construction of dataset standards, management, integration, usage, reuse, creation, and comparison of data elements in the biomedical field, and designs the knowledge model and the knowledge graph of the biomedical dataset, thereby realizing fine-grained decomposition and semantic enrichment of biomedical dataset standards. The knowledge model may include, but not limited to, a total of 21 entity types and 30 relationship types, which may be further extended based on specific needs to establish fine-grained associations between different types of standards, content units, and resources, as well as to determine a degree of association between specific entities.

The foregoing description of the disclosed embodiments enables a person skilled in the art to realize or use the present invention. Multiple modifications to these embodiments will be apparent to those skilled in the art, and the general principles defined herein may be realized in other embodiments without departing from the spirit or scope of the present invention. Accordingly, the present invention will not be limited to these embodiments shown herein, but will be subject to the broadest possible scope consistent with the principles and novel features disclosed herein. The basic concepts have been described above, and it will be apparent to those skilled in the art that the foregoing detailed disclosure is intended to be exemplary only, and does not constitute a limitation of the present disclosure. While not expressly stated herein, a person skilled in the art may make various modifications, improvements, and amendments to the present disclosure. Those types of modifications, improvements, and amendments are suggested in the present disclosure, so those types of modifications, improvements, and amendments remain within the spirit and scope of the exemplary embodiments of the present disclosure.

What is claimed is:

1. A method for automated processing of biomedical data, performed by a computing system including a processor and a memory, comprising:
determining, based on a knowledge graph, at least one candidate synonym pair from a biomedical dataset to be analyzed, wherein a topological distance between any two data elements of the at least one candidate synonym pair in the knowledge graph is less than or equal to a topological distance threshold;
determining an original context of data elements in the at least one candidate synonym pair based on the biomedical dataset to be analyzed;
performing a concurrent retrieval in a biomedical corpus based on the data elements, to recall at least K groups of context fragments distributed across different application scenarios for each of the data elements;
inputting the original context and the at least K groups of context fragments of the data elements included in the at least one candidate synonym pair into a large language model (LLM) to obtain a semantic consistency score that measures a semantic similarity degree of the at least one candidate synonym pair, wherein the LLM is a machine learning model;
determining a merged semantic group that is semantically equivalent from the at least one candidate synonym pair based on the semantic consistency score, and generating a merged semantic label for the merged semantic group;
dynamically deploying the merged semantic label into a data reading plug-in located between different biomedical systems; and
controlling the data reading plug-in to perform a process including:
in response to receiving a heterogeneous service data stream containing a heterogeneous character identifier, performing a standardized payload conversion on the heterogeneous service data stream based on the merged semantic label:
mapping the heterogeneous character identifier to a unified merged semantic label and appending the unified merged semantic label to a data packet; and
retaining the heterogeneous character identifier as an original traceability field.

2. The method according to claim 1, further comprising:
determining a node density and a connection complexity of an ontology category corresponding to each of the data elements based on the knowledge graph; and
determining a topological distance threshold corresponding to the data element based on the node density and the connection complexity, wherein data elements of different ontology categories have different topological distance thresholds.

3. The method according to claim 1, wherein the concurrent retrieval includes a sparse retrieval and a dense retrieval; and
the performing a concurrent retrieval in a biomedical corpus based on the data elements includes:
performing the sparse retrieval on the biomedical corpus to obtain a sparse fragment set;
constructing a data element vector and a corpus vector based on the data elements and a corpus text;
performing the dense retrieval based on the data element vector and the corpus vector to obtain a dense fragment set; and
performing a fusion ranking based on the sparse fragment set and the dense fragment set to recall the at least K groups of context fragments for each of the data elements.

4. The method according to claim 3, further comprising:
for each candidate synonym pair,
determining a topological distance set of data elements in the candidate synonym pair in the knowledge graph based on the knowledge graph; and
determining a number K of groups of context fragments required to be recalled for each of the data elements in the candidate synonym pair based on the topological distance set.

5. The method according to claim 3, wherein the performing a fusion ranking includes:
clustering the sparse fragment set and the dense fragment set based on the different application scenarios to obtain at least one scenario cluster;
for each scenario cluster of the at least one scenario cluster, sorting fragments within the scenario cluster according to a similarity between each of the fragments and a corresponding data element;
determining fragments to be merged from a sorting result of each scenario cluster based on a preset selection ratio; and
combining the fragments to be merged from all scenario clusters to obtain the context fragments.

6. The method according to claim 1, wherein the determining a merged semantic group that is semantically equivalent from the candidate synonym pair based on the semantic consistency score includes:
in response to the semantic consistency score of any candidate synonym pair being within a fuzzy interval, obtaining an associated feature of the data elements corresponding to the candidate synonym pair based on the knowledge graph; and
determining whether the candidate synonym pair is the merged semantic group based on the associated feature.

7. The method according to claim 1, further comprising:
dynamically deploying the merged semantic label into a control element of a sorting manipulator arm, and controlling the control element to perform a process including:
determining a target transport track for a sample to be sorted based on the merged semantic label and a character identifier of the sample to be sorted; and
controlling the sorting manipulator arm to transfer the sample to be sorted to the target transport track.

8. The method according to claim 1, further comprising:
obtaining structured data and an unstructured text of each multivariate standard text in a multi-source standard text set;
extracting an entity type and attribute data from the structured data and the unstructured text;
performing knowledge fusion on the entity type and the attribute data based on a semantic associative relationship to generate the knowledge graph;
wherein the semantic associative relationship includes: an inter-standard data relationship, a relationship between a data standard and a data element set, a relationship between a data element set and a data element, a relationship between a data element and a data element concept, a relationship between a data element and a value domain, a relationship between data elements, and a relationship between a dataset standard or a data element and a medical scale or a questionnaire;
the relationship between data elements is determined based on a Jaccard similarity between the data elements, wherein the Jaccard similarity between the data elements is determined based on the following formula:
$$Sim\_ele ( E 1 , E 2 ) = \frac{\vert A ∩ B \vert}{\vert A \vert + \vert B \vert - \vert A ∩ B \vert}$$
wherein E1 and E2 respectively denote two data elements, a text of each of the two data elements is tokenized, E denotes a tokenized text composed of a data element name and a data element definition of each of the two data element, Sim_ele_name( ) denotes a data element similarity, A denotes the tokenized text of E1, B denotes the tokenized text of E2, and a final similarity result is controlled within a range of [0, 1].

9. The method according to claim 8, wherein the obtaining structured data and an unstructured text of each multivariate standard text in a multi-source standard text set includes:
performing optical character recognition (OCR) on the multivariate standard text to obtain the structured data and the unstructured text.

10. The method according to claim 8, wherein the extracting an entity type and attribute data includes:
parsing the structured data and the unstructured text based on natural language processing (NLP) to extract the entity type and the attribute data.

11. The method according to claim 8, further comprising:
constructing an entity attribute table and a triple relationship table, batch converting data into Unicode Transformation Format-8-bit encoding (UTF-8 encoding), and importing the data into a graph database for storage; and
after importing the data, verifying correctness of triple data through sampling to ensure accuracy of the entity type and an associated relationship.

12. The method according to claim 10, further comprising:
identifying and extracting a data element text of the multivariate standard text;
performing data cleansing, review, and quality control on the data element text;
writing a regular expression according to a coding rule to perform identifier spell check and unified correction on the data element text; and
supplementing and modifying problems existing in the data element text, including a recognition error, a garbled text, or an omission, to form preliminary structured data.

13. The method according to claim 10, wherein the parsing the structured data and the unstructured text based on natural language processing (NLP) to extract the entity type and the attribute data includes:
identifying, extracting, and labeling the entity type and the unstructured text through a field lexicon or a machine learning approach.

14. The method according to claim 8, further comprising:
unifying entity data and types in the biomedical dataset to be analyzed based on a preset standard code and a name;
performing entity merging on data element texts of the multivariate standard text through similarity calculation; and
merging and disambiguating a value domain table name, and unifying the value domain table name in combination with a standard number.
